(19) United States
(12) Patent Application Publication
(10) Pub. No.: US 2012/0157844 A1
(43) Pub. Date: Jun. 21, 2012

(54) SYSTEM AND METHOD TO ILLUSTRATE ULTRASOUND DATA AT INDEPENDENT DISPLAYS

(75) Inventor: Menachem Halmann, Wauwatosa, WI (US)

(73) Assignee: GENERAL ELECTRIC COMPANY, Schenectady, NY (US)

(21) Appl. No.: 12/970,418

(22) Filed: Dec. 16, 2010

(51) Int. Cl. A61B 8/14

(52) U.S. Cl. 600/443

(57) ABSTRACT

An ultrasound imaging system is provided that comprises a beamformer, a first processor, and a second processor. The beamformer receives an ultrasound image data acquired by a transducer probe. The first processor processes the ultrasound image data communicated from the beamformer so as to create a first illustration at a first interface to show to a user of the ultrasound imaging system. The second processor processes the ultrasound image data communicated from the beamformer so as to create a second illustration at a second interface to show to a patient of the ultrasound imaging system. The first processor performs different processing steps compared to the second processor such that the first illustration at the first interface is different than the second illustration at the second interface.

### BACKGROUND

This invention generally relates to a method of and system for displaying ultrasound data, and more particularly to a system and method of illustrating ultrasound data on multiple visual displays.

Ultrasound imaging is a medical imaging technique for imaging organs and soft tissues in a human body. Ultrasound imaging uses real time, non-invasive high frequency sound waves to produce a two-dimensional (2D) image. Although, ultrasound imaging provides less anatomical information compared to CT or MRI, ultrasound imaging has several advantages in that patients are not exposed to radiation, studies of moving structures may be provided in real-time, the image scan is quickly performed and is inexpensive.

Conventional ultrasound imaging systems are known to include a dedicated monitor for displaying some combination of functional data (e.g., alphanumeric, physiological, real-time anatomical image data) associated with imaging during a medical procedure. A drawback of conventional ultrasound imaging systems is that certain functional data is desired for a physician or clinician to perform the medical procedure but may not be desired for display to a patient undergoing the medical procedure.

Hence there exists a need to provide a method of and system for illustrating graphic images that is readily interchangeable for a respective clinician or medical procedure.

### BRIEF DESCRIPTION

The above-mentioned shortcomings, disadvantages and problems are addressed by the embodiments described herein in the following description of a method and system of illustrating ultrasound image data between a physician or clinician versus that shown to the patient.

In one embodiment of the subject matter described herein, an ultrasound imaging system is provided. The system comprises a beamformer, a first processor, and a second processor. The beamformer receives an ultrasound image data acquired by a transducer probe. The first processor processes the ultrasound image data communicated from the beamformer so as to create a first illustration at a first interface to show to a user of the ultrasound imaging system. The second processor processes the ultrasound image data communicated from the beamformer so as to create a second illustration at a second interface to show to a patient of the ultrasound imaging system. The first processor performs different processing steps compared to the second processor such that the first illustration at the first interface is different than the second illustration at the second interface.

In another embodiment of the subject matter described herein, a method is provided, comprising the acts of receiving an ultrasound image data acquired by a transducer probe of an ultrasound imaging system; communicating the ultrasound image data to both a first processor and a second processor; processing the ultrasound image data by the first processor so as to create a first illustration at a first interface to show to a user of the ultrasound imaging system; and processing the ultrasound image data by the second processor so as to create a second illustration at a second interface to show to a patient of the ultrasound imaging system. The ultrasound image data is acquired by a beamformer of the ultrasound imaging system, and the first processor performs different processing steps compared to the second processor such that the first illustration at the first interface is different than the second illustration at the second interface.

In yet another embodiment of the subject matter described herein, an ultrasound imaging system is provided. The system comprises a beamformer to receive an ultrasound image data acquired by a transducer probe, a first processor to process the ultrasound image data communicated from the beamformer so as to create a first illustration at a first interface to show to a user of the ultrasound imaging system, and a second processor to process the ultrasound image data communicated from the beamformer so as to create a second illustration at a second interface to show to a patient of the ultrasound imaging system. The first processor performs different processing steps compared to the second processor such that the first illustration at the first interface is different than the second illustration at the second interface, and processing by the first processor includes increasing a contrast of image data illustrating a needle inserted into the patient, and wherein the processing by the second processor does not perform processing to increase the contrast of image data illustrating the needle in the creating the second illustration to show the patient.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a schematic diagram of an embodiment of a system in accordance to the subject matter described herein.

FIG. 2 shows a schematic diagram of an embodiment of a method of operating the system of FIG. 1 in accordance to the subject matter described herein.

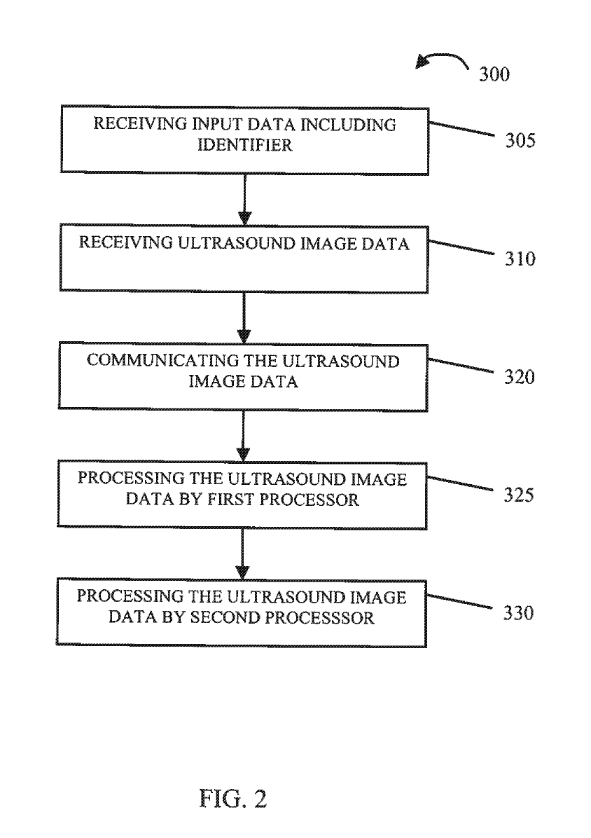

### DETAILED DESCRIPTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

In this document, the terms “a” or “an” are used, to include one or more than one. In this document, the term “or” is used to refer to a nonexclusive or, unless otherwise indicated.

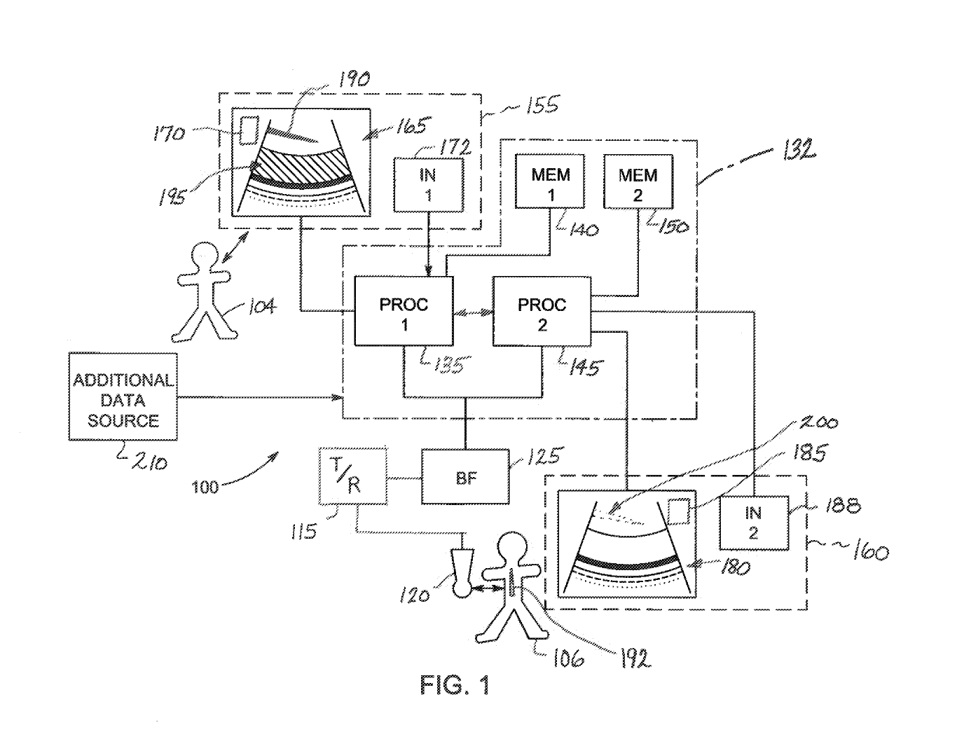

FIG. 1 illustrates an embodiment of an ultrasound imaging system 100 having a technical effect to simultaneously provide separate displays of general real-time, acquired ultrasound image data to the physician 104 and the imaged subject or patient 106, so as to optimally address their specific needs, in accordance to the subject matter described herein.

One embodiment of the ultrasound imaging system 100 is generally operable to acquire real-time ultrasound imaging data of the patient 106. Generally, the ultrasound imaging system 100 can include a transmitter/receiver 115 that drives an array of elements, for example, piezoelectric crystals, within a transducer, transducer probe or probe 120 to emit pulsed ultrasonic signals into a body or volume of an imaged subject 122. A variety probes 120 and geometries transmitting the ultrasound signals from the probe may be used. The ultrasonic signals are back-scattered from anatomical structures in the patient 106, for example, blood vessels or muscular tissue, to produce echoes that return to the elements of the probe 120 and received at the transmitter/receiver 115. The transmitter/receiver 115 communicates detection of the back-scattered ultrasound signals to the beamformer 125. The beamformer 125 generally performs beamforming including translating the echo data detected by the elements of the transducer 120 into ultrasound detection signal (e.g., RF). The beamformer 125 provides the ultrasound detection signal to a controller 130.

An embodiment of the controller 130 can generally include a first processor 135 in communication with a first memory 140, and a second processor 145 in communication with a second memory 150, that both in combination are operable to process and translate the ultrasound detection signal (e.g. RF signal or IQ data pairs) into a general real-time ultrasound image data for illustration. Each of the processors 135, 145 can be in communication to execute computer-readable program instructions stored in the memory 140 and 150, respectively, of the controller 130 to perform translation of the ultrasound detection signal into an ultrasound image data using different processing steps as according to the computer programmable instructions in the memory 140, 150 for execution by the processors 135, 145, respectively. Each of the processors 135, 145 can be instructed to perform one or more processing operations according to multiple selectable ultrasound modalities on the acquired ultrasound detection information. Acquired ultrasound detection information may be processed in real-time during a scanning session as the echo signals are received. Additionally or alternatively, the ultrasound detection information may be stored temporarily in either memory 140 and 150 during a scanning session and processed in less than real-time in a live or off-line operation. The acquired ultrasound detection data or information or signal not scheduled to for display can immediately be stored in either memory 140 and 150. The memory 140 or memory 150 may comprise any known data storage medium, for example, a permanent storage medium, removable storage medium, and the like.

The first processor 135 can be in wired or wireless communication with a first user interface 155 for visualization and interaction with the clinician or user 104, and provide certain control operations and be configured to receive inputs from the operator or user 104 of the system 100. In a like manner, the second processor 145 can be in wired or wireless communication with a second user interface 160 for illustration to the patient 106.

An embodiment of the first interface 155 can include one or more monitors that present a graphic display 165 of patient information, including diagnostic ultrasound images to the user 104 for review, measurement, diagnosis and analysis. At least a portion of the interface 155 can include a user selectable element 170 with touch sensitive portion or touch sensitive technology to receive input from the user 104, such as measurements or patient data. The interface 155 may automatically display the generated ultrasound image data in various formats as output from the processor 135, for example, planes from two-dimensional (2D) and/or three-dimensional (3D) ultrasound data either in real-time or from stored 2D or 3D data-sets of ultrasound detection or image data in the memory 140. The processing of the ultrasound detection or image data by the processor 135 can be based in part on user inputs, for example, user selections received at the user interface 155. The first interface 155 may further include input devices 172 such as a keyboard, a touch-screen, a keypad, a joystick, dials, or other conventional input device or combination thereof operable to receive data from the user or clinician for communication to the first processor 135 or memory 140.

An embodiment of the second interface 160 can include one or more monitors that present a graphic display 180 of diagnostic ultrasound images to the patient 106. At least a portion of the interface 160 can include a patient selectable element 185 with touch sensitive portion or touch sensitive technology to receive input from the patient 106, such as desire to download real-time ultrasound image data to a removable storage medium (e.g., CD, DVD, memory stick, etc.), or to automatically download the display 180 for communication in an email or to post to a social network website. The second interface 160 may automatically display the generated ultrasound image data in various formats as generated by the processor 145, for example, planes from two-dimensional (2D) and/or three-dimensional (3D) ultrasound data either in real-time or from stored 2D or 3D data-sets of ultrasound detection or image data in the memory 140. The second interface 160 may further include input devices 188 such as a keyboard, a touch-screen, a keypad, a joystick, dials, or other conventional input device or combination thereof operable to receive data from the patient 106 for communication to the processor 145 or memory 150.

In general real-time, the processor 135 can be configured to process the acquired ultrasound image data from the beamformer 125 to create a first display 165 to the user 104 simultaneously in general real-time with the execution of the processor 145 to process the acquired ultrasound image data from the beamformer 125 to create a second display 180 to the patient 106. Yet, the processor 135 can be configured to process the acquired ultrasound image data from the beamformer 125 with different processing steps or to include different textual information, review, measurement, diagnosis and analysis than compared to the processing of the ultrasound image data by the processor 145. For example, the processor 135 may process the ultrasound image data in combination with illustration of graphically illustrated user tools, or prompts to or requests by the user at the interface 155. Examples of a user tool can include performing compounding or cross beam processing of the ultrasound image data in a manner to create an illustration 190 of a highlight of an invasive device such as a needle or probe 192 (e.g., needle or probe that can administer local/regional anesthesia drug, biopsy extraction tool) in contrast to illustration of tissue. Another example of a user tool can include an illustration of a measurement on a developing fetus in the interface 155 in contrast to tissue. Yet another example of user tool can be instructions to the processor 135 to create an illustration 195 of a highlight (see cross-hatch in graphic display 165) of a detected tumor in contrast to healthy tissue. These user tools described herein are known in the art and not described in detail.

The processor 145 can be configured to process the ultrasound image data in a different manner without including illustration of user tools or prompts to or requests by the user, in a manner that optimally addresses the specific needs of the patient 106 separate and independent of the needs of the user 104 in regard to user tools described above. For example, the processor 145 can be configured to process the acquired ultrasound image data from the beamformer without combination with user tools as described above. Thereby, the processor 145 communicates to create illustration of the ultrasound image at the interface 160 without showing the illustration 190 of the highlight of the invasive tool (or showing a standard illustration 200 of the invasive tool using standing image processing employed on the surrounding tissue, shown in dashed line in graphic display 180) or without showing the illustration 195 of the detected tumor. Thereby, the patient does not view these above-described user tools while still able to view illustration of the ultrasound image on the interface 160 in a desired manner. The processor 145 can also be configured to receive a patient instruction from user element 185 at the second interface 160 to automatically download and communicate the display 180 in an email or to share on a social network website.

Each of the user selectable elements 170 or 185 can be operable to receive input of a type of image processing or a desired setup of the type of illustration on each interface 155, 160 without application on processing image data for the other interface 160, 155, respectively.

The interfaces 155, 160 can also be connected in communication to receive input data or image data from another source 210. for combination with the illustration of ultrasound image data at the interfaces 155, 160. The additional data source 210 can include for example a Computed Tomography (CT) imaging system, a magnetic resonance imaging (MRI) imaging system, a electrocardiogram (ECG) system, a Positron Emission Transmission/Computed Tomography (PET/CT) imaging system, a second ultrasound imaging systems, a real-time fluoroscopic imaging system, an endoscopic imaging system, etc. The additional data sources 210 can be operable to generate a signal to create a visual or graphic illustration in combination with the ultrasound image data on the first display for viewing by the clinician 104 or on the second display for viewing by the patient 106. The additional data sources 210 can also include a workstation operable to receive and/or store one or more pre-recorded or real-time visual illustrations generated by the additional data source and stored for later access by the system 100. The additional data source 210 can be connected to communicate the visual illustration directly or indirectly to the first display or second display of the system 100. The visual illustration from the additional data source 210 can include, but is not limited to, a representation of a physiological waveform, an anatomical image, physiological functional image (e.g., ultrasound images, transesophagial ultrasound acquired image, transthoracic ultrasound acquired image, intravascular ultrasound (IVUS) acquired image, alphanumeric data or messages representative of measured data, a software interface or window, and other conventional medical acquired data, and/or combinations thereof) acquired in real-time, pre-recorded, continuous, periodic, or selected manner.

Embodiments of the controller 130 can be a stand-alone computer (e.g., desktop or laptop, blackberry, etc.) or can include various arrangements or combinations of various types of processors (e.g., microprocessor, programmable logic controller, etc.) or combinations thereof in communication with various types of memory or computer readable mediums (e.g., memory stick, hard-drive, disk, CD, DVD, or other conventional storage medium or combination thereof).

The interfaces 155 and 160 can also include output devices such as LCD or LED monitors, hand-held display, CRT projector, personal data assistant (PDA), LEDs lights, touch-screens, alarm devices, etc. Examples of touch-screen technology that can be provided on the interface 155, 160 can include but is not limited to touch sensitive elements such as capacitive sensors, membrane switches, and infrared detectors. Examples of touch-screen technology that can be provided on the interfaces 155 and 160 can include but is not limited to touch sensitive elements such as capacitive sensors, membrane switches, and infrared detectors.

The interfaces 155, 160 may automatically display the generated ultrasound image data in various formats as communicated from different processing steps from the processors 135, 145 respectively. For example, the formats can include planes from two-dimensional (2D) and/or three-dimensional (3D) ultrasound data either in real-time or from stored 2D or 3D data-sets of ultrasound detection or image data in the memory 140. The processing of the ultrasound detection or image data by the processor 135, 145 can be based in part on inputs at the interfaces 155, 160, respectively, for example, user selections received at the user interface 155.

The interface 155, 160 may further include input devices 172, 188 respectively operable to receive data from the user or clinician or patient 106, respectively for communication to the processor 135, 145 or memory 140, 150, respectively. An embodiment of the interface 155 can include the first input device 172 in communication with the first processor 135 and the second input device 188 in communication with the second processor 145. The input devices 172, 188 can include a keyboard, a touch-screen, a keypad, a joystick, dials, or other conventional input devices or combination thereof operable to receive data from the user or clinician 104 or the patient 106.

It should be understood that the number of interfaces 155, 160, and display fields 165, 180 and input devices 172, 188 associated therewith can vary, as well as their location e.g., a cart, a wall, a ceiling, etc. or combinations thereof and is not limiting.

Having described a general construction of the embodiment of the ultrasound imaging system 100, the following is a general description of a method of operation of the ultrasound imaging system 100 described above. Although the method 300 is described in accordance to the following acts, it should be understood that the sequence of the acts can vary. Also, it should be understood that the following description of acts is not limiting, and that one or more of the described acts may not be needed. It should be understood that each of the described acts can be representative of computer readable program instructions stored in the memory 140, 150 for execution by the processors 135 or 145, respectively.

Assume initially the user or clinician is operable to program the controller 130 via the input 172 at interface 155 with multiple preprogrammed arrangements of the first visual illustration of the acquired ultrasound image data in the first display simultaneous with the second visual illustration of the acquired ultrasound image data in the second display in accordance to at least a step of a medical procedure. Each of the predetermined arrangements of display fields/illustrations 165, 180 can be stored in the memory 140, 150 with an identifier either assigned by the user or operator 104 for access by the processors 135, 145. In one example, one or more of the multiple pre-programmed arrangements of display fields/illustrations 165, 180 can be stored with an identifier of a certain medical procedure. In yet another example, one or more of the multiple pre-programmed arrangements of display fields/illustrations 165, 180 can be stored with an identifier indicative of the user or clinician 104 to execute the medical procedure.

Act 305 includes the first interface receiving an input data including an identifier associated with one of a plurality of arrangements, wherein each arrangement includes a set of instructions to the first and second processors 135, 145 to perform the different processing steps to create the first and second display fields/illustrations 165, 180, respectively. The identifier can be indicative of a step of a medical procedure, or of a physician name.

Act 310 includes receiving an ultrasound image data acquired by a transducer probe of an ultrasound imaging system. Act 320 includes communicating the ultrasound image data to both a first processor and a second processor. Act 325 includes processing the ultrasound image data by the first processor 135 so as to create the first display field or illustration 165 at the first interface 155 to show to the user or operator 104 (e.g., physician, clinician) of the ultrasound imaging system 100. Act 330 includes processing the ultrasound image data by the second processor 145 so as to create the second display field or illustration 180 at a second interface 160 to show to the patient 106 of the ultrasound imaging system 100. The first and second display fields or illustrations 165, 180 can both or either include generally real-time acquired ultrasound image data communicated from the beamformer 125. The first processor 135 can performs different processing steps compared to the second processor 145 such that the first display field or illustration 165 at the first interface 155 is different than the second illustration 180 at the second interface 160, though each simultaneously display the same ultrasound image data acquired by the beamformer 125.

In one example, the processing by the first processor 135 includes increasing a contrast of image data 190 illustrating a needle or probe 192 inserted into the patient 106, and wherein the processing by the second processor 145 does not perform processing to increase a contrast of image data 200 illustrating the needle or probe 192 in the creating the second display field or illustration 180 to show the patient 106. In another example, the first ultrasound image data in the first display field or illustration 165 at the first interface 155 to the user or clinician 104 may be better illustrated in color, while the second ultrasound image data in the second display field or illustration 180 at the second interface 160 may be more suitable for illustration to the patient 106 in black and white. In yet another example, the visual illustration for the first display field/illustration 165 may be better illustrated at very higher refresh rate as performed by the first processor 135 compared to the visual illustration for the second display field/illustration 180 or the second display field/illustration 180 may be sufficiently illustrated at lower refresh rate as performed by the second processor 135 for illustration to the patient 106.

A technical effect of the described ultrasound imaging system 100 and method 300 provides a first illustration of real-time ultrasound imaging data to a user 104 while simultaneously providing a second illustration of real-time ultrasound imaging data to a patient 106 during a step of a medical procedure employing the ultrasound imaging system 100. One embodiment of the ultrasound imaging system 100 can be generally operable to acquire real-time ultrasound imaging data of the patient 106. The described ultrasound imaging system 100 comprises the beamformer to receive the ultrasound image data acquired by the transducer probe; the first processor to process the ultrasound image data communicated from the beamformer so as to create the first illustration at the first interface to show to the user of the ultrasound imaging system; the second processor to process the ultrasound image data communicated from the beamformer 125 so as to create the second illustration at the second interface to show to the patient of the ultrasound imaging system. The first processor 135 performs different processing steps compared to the second processor 145 such that the first display field/illustration 165 at the first interface 155 is different than the second display field/illustration 180 at the second interface 160. The method 300 describes processing by the first processor 135 to increase a contrast or otherwise enhance visualization of acquired ultrasound image data 190 illustrating a needle or probe 192 inserted into the patient 106, and wherein the processing by the second processor 145 does not perform processing to increase the contrast or otherwise enhance visualization of image data 200 illustrating the needle or probe 192 in the creating the second display field/illustration 180 to show the patient 106.

The first interface 155 is operable to receive an input data including an identifier associated with one of a plurality of arrangements, wherein each arrangement includes a set of instructions to the first and second processors to perform the different processing steps to create the first and second illustrations. The identifier can be indicative of a step of a medical procedure, or indicative of a physician name.

In various embodiments of the invention, the method of forming an ultrasound image as described herein or any of its components may be embodied in the form of the processor 135, 145. Typical examples of the processor 135, 145 include a general-purpose computer, a programmed microprocessor, a digital signal processor (DSP), a micro-controller, a peripheral integrated circuit element, and other devices or arrangements of devices, which are capable of implementing the steps that constitute the methods described herein.

As used herein, the term “processor” may include any computer, processor-based, or microprocessor-based system including systems using microcontrollers, reduced instruction set circuits (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The processors 135, 145 execute a set of computer readable instructions (e.g., corresponding to the method 300 described herein) that are stored in one or more memories (also referred to as computer usable medium) 140, 150. The memories 140, 150 may be in the form of a database or a physical memory element present in the processors 135, 145. The processor 135, 145 may also hold data or other information as desired or needed. The processor 135, 145 can be, for example, but not limited to, an electronic, magnetic, optical, electromagnetic, infrared, or semiconductor system, apparatus, device, or propagation medium. More specific examples of the processor 135, 145 include, but are not limited to, the following: a random access memory (RAM) a read-only memory (ROM), an erasable programmable read-only memory (EPROM or Flash memory), a Hard Disc Drive (HDD) and a compact disc read-only memory (CDROM).

The set of computer readable program instructions may include various commands that instruct the processing machine to perform specific operations such as the processes of the various embodiments of the invention. The set of computer readable program instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

In various embodiments of the subject matter describe herein, the method 300 can be implemented in software, hardware, or a combination thereof. The method 300 can be provided by various embodiments of the ultrasound imaging system 100, for example, can be implemented in software by using standard programming languages such as, for example, C, C++, Java, and the like.

As used herein, the terms “software” can include any computer program stored in memory for execution by the processors 135, 145, including RAM memory, ROM memory, EPROM memory, EEPROM memory, and non-volative RAM (NVRAM) memory. The above memory types are exemplary only, and are thus limiting as to the types of memory usable for storage of the computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An ultrasound imaging system, comprising:
a beamformer to receive an ultrasound image data acquired by a transducer probe;
a first processor to process the ultrasound image data communicated from the beamformer so as to create a first illustration at a first interface to show to a user of the ultrasound imaging system; and
a second processor to process the ultrasound image data communicated from the beamformer so as to create a second illustration at a second interface to show to a patient of the ultrasound imaging system,
wherein the first processor performs different processing steps compared to the second processor such that the first illustration at the first interface is different than the second illustration at the second interface.

2. The system of claim 1, wherein processing by the first processor includes increasing a contrast of image data illustrating a needle inserted into the patient, and wherein the processing by the second processor does not perform processing to increase the contrast of image data illustrating the needle in the creating the second illustration to show the patient.

3. The system of claim 1, wherein the first interface is operable to receive an input data including an identifier associated with one of a plurality of arrangements, wherein each arrangement includes a set of instructions to the first and second processors to perform the different processing steps to create the first and second illustrations.

4. The system of claim 3, wherein the identifier is indicative of a step of a medical procedure.

5. The system of claim 3, wherein the identifier is indicative of a physician name.

6. The system of claim 1, wherein at least one of the first and second illustrations includes generally real-time detected ultrasound image data communicated from the beamformer.

7. The system of claim 1, further comprising a user element on the second interface to receive an instruction from the patient to automatically download and communicate the second illustration to share on a social network.

8. A method comprising the acts of:
receiving an ultrasound image data acquired by a transducer probe of an ultrasound imaging system;
communicating the ultrasound image data to both a first processor and a second processor;
processing the ultrasound image data by the first processor so as to create a first illustration at a first interface to show to a user of the ultrasound imaging system; and
processing the ultrasound image data by the second processor so as to create a second illustration at a second interface to show to a patient of the ultrasound imaging system, wherein the ultrasound image data is acquired by a beamformer of the ultrasound imaging system, and wherein the first processor performs different processing steps compared to the second processor such that the first illustration at the first interface is different than the second illustration at the second interface.

9. The method of claim 8, wherein processing by the first processor includes increasing a contrast of image data illustrating a needle inserted into the patient, and wherein the processing by the second processor does not perform processing to increase the contrast of image data illustrating the needle in the creating the second illustration to show the patient.

10. The method of claim 8, wherein the first interface receives an input data including an identifier associated with one of a plurality of arrangements, wherein each arrangement includes a set of instructions to the first and second processors to perform the different processing steps to create the first and second illustrations, respectively.

11. The method of claim 10, wherein the identifier is indicative of a step of a medical procedure.

12. The method of claim 10, wherein the identifier is indicative of a physician name.

13. The method of claim 8, wherein at least one of the first and second illustrations includes generally real-time detected ultrasound image data communicated from the beamformer.

14. The method of claim 8, further comprising providing a user element on the second interface to receive an instruction from the patient to automatically download and communicate the second illustration to share on a social network.

15. An ultrasound imaging system, comprising:
a beamformer to receive an ultrasound image data acquired by a transducer probe;
a first processor to process the ultrasound image data communicated from the beamformer so as to create a first illustration at a first interface to show to a user of the ultrasound imaging system; and
a second processor to process the ultrasound image data communicated from the beamformer so as to create a second illustration at a second interface to show to a patient of the ultrasound imaging system,
wherein the first processor performs different processing steps compared to the second processor such that the first illustration at the first interface is different than the second illustration at the second interface, and
wherein processing by the first processor includes increasing a contrast of image data illustrating a needle inserted into the patient, and wherein the processing by the second processor does not perform processing to increase the contrast of image data illustrating the needle in the creating the second illustration to show the patient.

16. The ultrasound imaging system, further comprising a user element on the second interface to receive an instruction from the patient to automatically download and communicate the second illustration to share on a social network.
